(19) United States
(12) Patent Application Publication
(10) Pub. No.: US 2016/0369219 A1
(43) Pub. Date: Dec. 22, 2016

(54) SYSTEM AND METHOD FOR PRODUCING BIOGAS

(71) Applicant: GEA FARM TECHNOLOGIES CANADA INC., Drummondville (CA)

(72) Inventor: Alain Courtemanche, Lefebvre (CA)

(21) Appl. No.: 14/443,366

(22) PCT Filed: Nov. 18, 2013
(86) PCT No.: PCT/CA2013/050879

§ 371 Date: May 15, 2015

Related U.S. Application Data
(60) Provisional application No. 61/727,208, filed on Nov. 16, 2012.

(51) Int. Cl. C12M 1/00; C02F 11/04; C12M 1/06; F28D 20/00; C12P 5/02; C12M 1/107

(52) U.S. Cl. CPC C12M 27/20; C12P 5/023; C12M 21/04; C12M 27/02; C12M 23/02; C12M 23/36; F28D 20/0034; C02F 11/04; C02F 2103/20

(57) ABSTRACT

A system (10) and method for producing biogas (16) from biomass (14). The system (10) includes a digestion reservoir (20) having a path defined by passageways (32) along which biomass (14) is conveyed and digested, the digestion reservoir (20) being configured for containing biogas (16) generated from a digestion of biomass (14) along the path. The system (10) also includes at least one mixing assembly (40) located along the path, each mixing assembly (40) being operatively connected between different passageway segments of the path so as to be able to selectively mix given biomass (14) from one segment to another, in order to increase overall production of biogas (16) along the path.

### FIELD OF THE INVENTION

The present invention relates to biogas production. More particularly, the present invention relates to a system and method for producing biogas from decaying organic matter.

### BACKGROUND OF THE INVENTION

It is known to generate a biogas, typically made up of methane, from the decomposition of biomass. This process can be referred to as “biomass digestion”.

As the biomass decomposes, the biogas is emitted and recuperated. Often, the recuperated biogas is combusted in a gas turbine so as to generate electrical energy. Although such a system of generating electrical energy has been demonstrated, it has been shown to have some of the following disadvantages: a) it requires a significant initial investment in the required equipment and is thus often dependent on governmental support; b) the technical skills needed to control, adjust and repair such a system is not in the normal field of competence of a lay person, such as a farmer (for example), who may supply the biomass, which results in increased maintenance and labour cost; c) there can be significant losses of energy during the generation of electricity from the biogas; d) etc.

Indeed, the energy losses mentioned in point c) above can occur during the transformation of the biomass from chemical to electrical energy, and ultimately reduce the amount of final usable energy. Firstly, to generate electrical energy, biogas must be combusted in the combustion chamber of the gas turbine. As the burning efficiency is often less than 100%, a first energy loss occurs. Secondly, the combusted biogas expands and pushes against a turbine/piston. At least some of the energy released by the combusted biogas is used up to move these components and to overcome mechanical friction, thereby resulting in a second energy loss. Thirdly, some of the components of the turbine are heated by the combustion of the biogas, and this heat is thus not usable energy, resulting in a third energy loss. Fourthly, the generator and control devices of the turbine provide electrical and mechanical resistance which must be overcome, thus resulting in a fourth energy loss. Finally, the electrical energy generated often must be consumed immediately, often cannot be stored, and/or can only be stored temporarily at great inefficiency, resulting in a fifth energy loss.

Some of the known systems used for generating biogas are described in the following American patents or patent applications known to the Applicant: U.S. Pat. Nos. 4,302,329; 4,350,588; 4,396,402; 4,927,530; 5,091,315; 5,096,579; 5,560,819; 6,342,378 B1; 6,395,173 B1; 6,451,589 B1; 6,521,129 B1; 6,613,562 B2; 6,783,677 B1; 6,811,701 B2; 6,841,072 B2; 6,982,035 B1; 7,078,229 B2; 7,179,642 B2; 7,608,439 B2; 7,641,796 B2; 7,727,396 B1; 7,820,429 B2; 7,951,296 B2; 7,993,521 B2; 8,202,721 B2; 8,298,424 B2; 8,318,997 B2; 8,394,271 B2; 2003/0111410 A1; 2004/0108267 A1; 2004/0154982 A1; 2006/0065593 A1; 2006/0289356 A1; 2007/0029243 A1; 2008/0138885 A1; 2008/0277336 A1; 2009/0123965 A1; 2009/0162914 A1; 2009/0218279 A1; 2009/0227003 A1; 2009/0280557 A1; 2009/0305376 A1; 2009/0305379 A1; 2010/0105128 A1; 2010/0151552 A1; 2010/0233778 A1; 2011/0042305 A1; 2011/0180633 A1; 2011/0226440 A1; 2011/0228633 A1; 2011/0256603 A1; 2011/0275141 A1; 2011/0281254 A1; 2012/0009668 A1; 2012/0329139 A1; 2013/0029410 A1; 2013/0095546 A1; 2013/0095561 A1; 2013/0133386 A1; 2013/0146533 A1; and 2013/0183752 A1.

Also known to the Applicant are the following documents: CA 1,198,605; DE 10 2007 024 947 A1; DE 20 2010 015 332 U1; WO 2013/039407 A1; and WO 2013/144703 A1.

In general, a first type of system for generating biogas, which can be referred to as an “infinitely mixed” system, typically consists of a large tank made of concrete which contains the digesting biomass (manure, for example). The tank is covered by a leak-tight dome which prevents the generated biogas from escaping, and provides a volume in which the biogas accumulates over the digesting biomass. The biomass often must be lightly agitated to facilitate the bacterial reaction and so as to avoid creation of a top solid layer of dried biomass that would stop biogas emission. Such a system often requires 30 days to complete biomass digestion, and digested biomass must be evacuated at the end of the cycle and replaced by new biomass. Although such a system allows for the bacteria digesting the biomass at the end of the cycle to contact the new biomass to be digested and thus to activate the reaction on that new biomass, it can include some of the following disadvantages:

a) the new entering biomass is often mixed without control with old exiting biomass which is at the end of the digesting process, which can result in new biomass being expelled from the system and old biomass that has already been digested being kept in the system, and thus reducing biogas generation for a given volume of digesting biomass;

b) whenever maintenance or replacement needs to be performed, such as on the agitators of the biomass, for example, the system is stopped, accumulated biogas is evacuated to atmosphere, and digested manure is returned to the pit from whence it came. At this time, electrical energy production is stopped for many weeks, and system reboot requires roughly 60 days, which can correspond to the time needed to fill the tank again and reactivate the digestion reaction with bacteria, thereby reducing the amount of usable energy generated by the system, and also profitability; and

c) digesting biomass often must be maintained around 100° F. to obtain the optimal reaction speed and quantity of biogas. Some energy must thus be injected into the biomass to maintain this temperature. This energy can represent around 25% of the total energy generated by the biogas system. Many systems of the prior art use the heat from the turbine along with a heat exchanger to pre-heat arriving new biomass before its addition in the tank. Such components often require maintenance and cleaning, and require the pumping and circulation of heated fluid, all of which increase complexity and reduce the final usable electric energy.

Another such system used for generating biogas, commonly referred to as a “plugged flow” system, often consists of a long tunnel. New biomass enters the tunnel, and pushes against the older biomass at the end of digesting process, which is expelled from the tunnel at the same rate as the arrival of the new biomass. The size and length of the tunnel can be calibrated with the daily manure production volume of the farm, or the input quantity for other processes, so as to obtain a processing time of about 30 days. A light tumbling effect is applied to avoid creation of a solid crust on the top of the biomass that would block biogas emission. Although such a system can allow for control of the digesting time of biomass in the tunnel because new biomass pushes against older biomass and thus forces the older biomass out the end of the tunnel, it can include some of the following disadvantages: a) the digestion reaction starts slowly because there is no mixing between the old biomass replete with bacteria and the new biomass which has much less, thus forcing the bacteria to migrate from old biomass to new; and b) the system does not address the maintenance and energy consumption disadvantages discussed above with respect to the “infinitely mixed” system.

Hence, in light of the above, there is a need for an improved system or method which, by virtue of their design, components and/or operating steps, would be able to overcome or at least minimize some of the aforementioned prior art disadvantages.

### SUMMARY OF THE INVENTION

An object of the present invention is to provide a system which satisfies some of the above-mentioned needs and which is thus an improvement over other related systems and/or methods known in the art.

According to the present invention, there is provided a system for producing biogas from biomass, the system comprising:

a digestion reservoir having a path defined by passageways along which biomass is conveyed and digested, the digestion reservoir having an inlet fluidly connected to the path for receiving biomass to be digested, and an outlet fluidly connected to the path for releasing digested biomass, the digestion reservoir being further configured for containing biogas generated from a digestion of biomass along the path; and

at least one mixing assembly located along the path, each mixing assembly being operatively connected between different passageway segments of the path so as to be able to selectively mix given biomass from one segment to another, in order to increase overall production of biogas along the path.

Other possible aspects, embodiments, variants and/or resulting advantages of the present invention, these being preferred and/or optional, are briefly described hereinbelow.

For example, according to a given possible embodiment, the path can be an internal path to the digestion reservoir, and the biomass can be agricultural manure provided by at least one neighboring farm, the manure displaying “fluid-like” behavior so as to be pumped into the system for processing (ex. digesting, biogas production, etc.), although various other types of biomass are contemplated by the present system, as will be explained hereinbelow.

According to another aspect of the present invention, there is also provided a system for producing biogas from biomass, the system comprising:

a digestion reservoir having a path along which biomass is conveyed and digested, the digestion reservoir having an inlet fluidly connected to the path for receiving biomass to be digested, and an outlet fluidly connected to the path for releasing digested biomass, the digestion reservoir being further configured for containing biogas generated from a digestion of biomass along the path; and

at least one mixing assembly located along the path, each mixing assembly being operatively connected between upstream and downstream passageways of the path so as to be able to selectively transfer given biomass from one passageway to another, in order to increase overall production of biogas along the path.

According to yet another aspect of the present invention, there is also provided a system for producing biogas from decaying organic matter, the system comprising:

a reservoir for collecting the biogas, the reservoir comprising an inlet for receiving organic matter to be processed and an outlet for conveying decayed organic matter, organic matter being conveyed from the inlet to outlet; and

at least one vertical divider affixed to a base of the reservoir and configured for dividing the reservoir into a first passageway and a second passageway extending at least partially alongside the first passageway, organic matter decaying and producing biogas while being conveyed from the inlet along the first and second passageways and towards the outlet.

In some optional configurations, the reservoir is sealed, and/or heated such as with circulating heated water, thereby maintaining the organic matter at a certain temperature range. Optionally, the organic matter is agricultural manure, such as liquid manure.

The at least one vertical divider can be many vertical dividers, each vertical divider defining another passageway. For example, there can be one single vertical divider dividing the reservoir into two passageways. Alternatively, there can be two vertical dividers dividing the reservoir into three passageways. Alternatively also, there can be three vertical dividers dividing the reservoir into four passageways, etc. The vertical dividers can include an agitation unit for agitating the organic matter as it is conveyed. The agitation unit can also transfer decayed and/or partially decayed organic matter from one passageway to non-decayed organic matter in a preceding passageway.

The system can also include an energy storage unit. The energy storage unit can be a volume of liquid, such as water, contained within a suitable container, which stores the heat produced from burning the biogas. Optionally, the energy stored in the energy storage unit can be used wherever a reliable source of heat is required, such as in a greenhouse, for example.

According to another aspect of the present invention, there is provided a method for producing biogas from biomass (ex. organic matter), the method comprising the steps of:
a) conveying and digesting biomass in a digestion reservoir along a path defined by a plurality of passageways; and
b) transferring or mixing given biomass from one passageway to another in order to increase overall production of biogas along the path of the digestion reservoir.

The method may comprise the step of agitating biomass at discrete locations along the path.

The method may also comprise the step of introducing beneficial bacteria into the digestion reservoir.

The method may also comprise the step of regulating the temperature of the digestion reservoir, and/or of other basins used therewith.

The method may also comprise the step of maintaining the biomass at a temperature of about 100° F.

The above-mentioned step b) may comprise the step of digesting biomass during a period of about 30 days.

According to another aspect of the present invention, there is provided a method for producing biogas from decaying organic matter, the method comprising the steps of:
a) introducing the organic matter to a reservoir; and
b) displacing the decaying organic matter within the reservoir in adjacent passageways, the decaying organic matter producing biogas while being displaced.

In some optional configurations, the method also includes the steps of introducing beneficial bacteria into non-decayed organic matter. Such an introduction of bacteria can be accomplished by agitating and/or transferring the organic matter, as described above.

According to another aspect of the present invention, there is provided a fluid circuit provided with the above-mentioned system(s) and/or components thereof.

According to another aspect of the present invention, there is provided an facility (ex. farm, plant, etc.) provided with the above-mentioned system(s), fluid circuit and/or components thereof.

According to another aspect of the present invention, there is provided a method of installing (i.e. assembling) and/or operating the above-mentioned system(s), fluid circuit and/or facility.

According to another aspect of the present invention, there is provided a kit with corresponding components for assembling the above-mentioned system(s) and/or fluid circuit.

According to yet another aspect of the present invention, there is also provided a method of assembling components of the above-mentioned kit.

According to yet another aspect of the present invention, there is also provided a fluid (i.e. biogas) having been produced with the above-mentioned kit, system(s), fluid circuit, facility and/or method(s).

The objects, advantages and other features of the present invention will become more apparent upon reading of the following non-restrictive description of optional embodiments thereof, given for the purpose of exemplification only, with reference to the accompanying drawings.

### BRIEF DESCRIPTION OF THE DRAWINGS

FIG. 1 is top plan view of a system for producing biogas, according to an optional configuration of the present invention.

FIG. 2 is an exposed side elevational view of a reservoir being shown with organic matter and biogas, according to an optional configuration of the present invention.

FIG. 3 is top plan view of the system of FIG. 1 being shown provided with greenhouses for receiving heat byproducts.

FIGS. 4a, 4b and 4c provide side elevational views of the system of FIG. 1.

FIGS. 5a, 5b and 5c provide close-up views of certain components shown in FIG. 4.

FIG. 6 is an enlarged view of a portion of what is shown in FIG. 1.

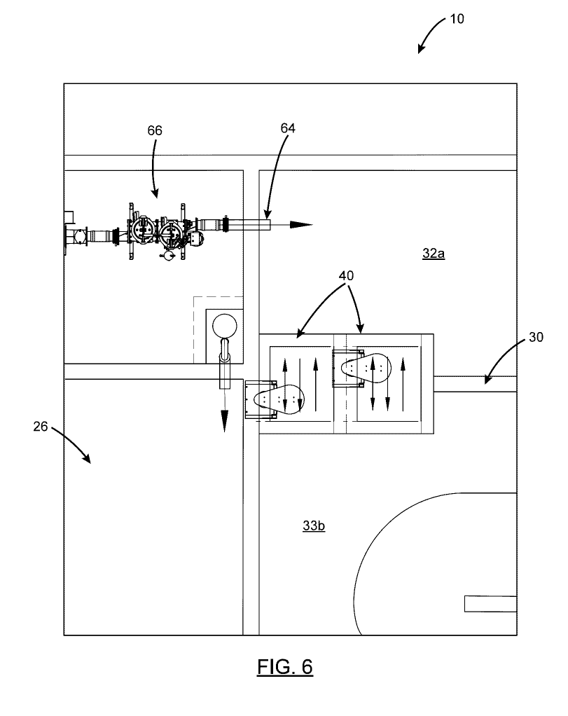

FIG. 7 is an enlarged view of another portion of what is shown in FIG. 1.

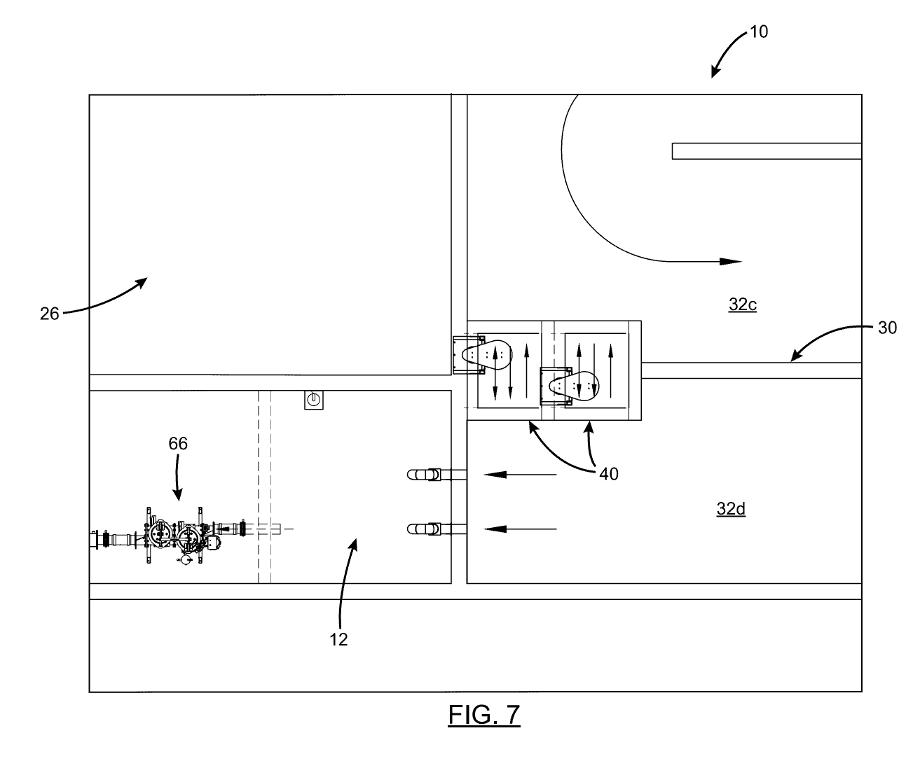

FIG. 8 is a perspective view of a wall arrangement of a reservoir according to an optional configuration of the present invention.

FIG. 9 is another perspective view of what is shown in FIG. 8.

FIG. 10 is a facility provided with a system for producing biogas, according to an optional configuration of the present invention, including the wall arrangement of FIG. 8 being now shown covered by outer portions of the facility.

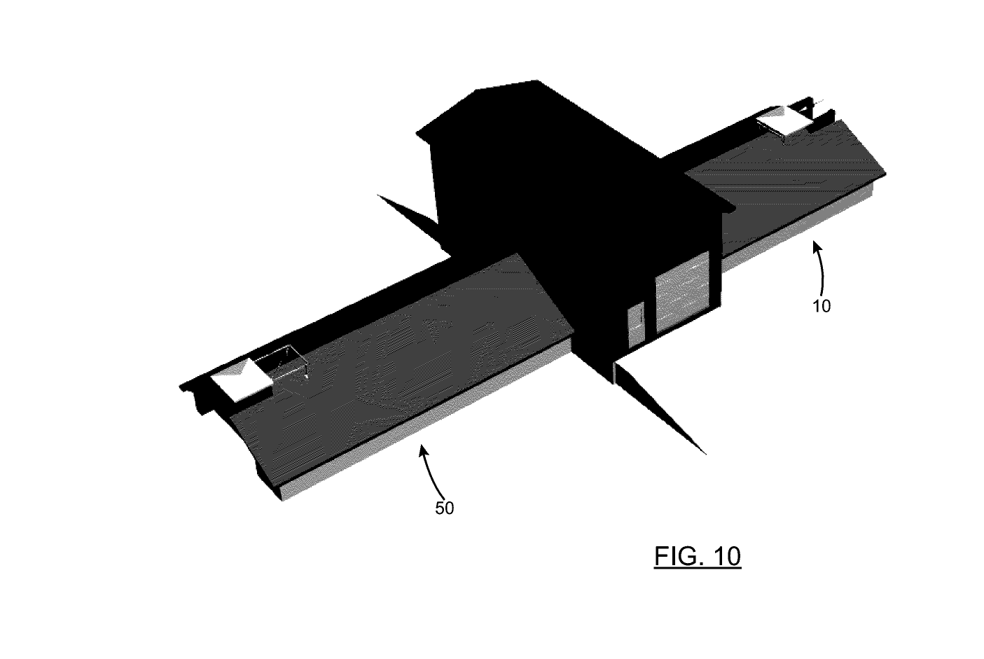

### DETAILED DESCRIPTION OF OPTIONAL EMBODIMENTS OF THE INVENTION

In the following description, the same numerical references refer to similar elements. The embodiments, geometrical configurations, materials mentioned and/or dimensions shown in the figures or described in the present description are optional embodiments only, given for exemplification purposes only.

Moreover, although the present invention was primarily designed for generating biogas from organic matter, it may be used with other types of matter and objects, and in other fields and for other purposes. For this reason, expressions such as “biogas”, “biomass”, “organic matter”, etc., used herein should not be taken as to limit the scope of the present invention and includes all other kinds of objects or fields with which the present invention could be used and may be useful.

Moreover, in the context of the present invention, the expressions “system”, “kit”, “plant”, “reservoir”, “device”, “digester”, “assembly”, “station” and “unit”, as well as any other equivalent expressions and/or compound words thereof will be used interchangeably. This applies also for any other mutually equivalent expressions, such as, for example: a) “fluid”, “gas”, “liquid”, “slurry”, “supply”, “manure”, etc.; b) “biomass”, “organic matter”, etc.; c) “decaying”, “decomposing”, “producing”, “generating”, “promoting, “increasing”, etc.; d) “agitating”, “perturbing”, “mixing”, “transferring”, “displacing”, “pumping”, etc.; e) “new”, “non-digested”, “upstream”, “less-digested”, etc.; f) “old”, “downstream”, “digested”, “more-digested”, etc.; g) “upper”, “top”, etc.; h) “lower”, “bottom”, etc.; and as well as for any other mutually equivalent expressions, pertaining to the aforementioned expressions and/or to any other structural and/or functional aspects of the present invention.

Furthermore, in the context of the present description, it will be considered that expressions such as “connected” and “connectable”, or “mounted” and “mountable”, may be interchangeable, in that the present invention also relates to a kit with corresponding components for assembling a resulting fully assembled system.

In addition, although the optional embodiments of the present invention as illustrated in the accompanying drawings may comprise various components, and although the optional embodiments of the system as shown consist of certain geometrical configurations as explained and illustrated herein, not all of these components and geometries are essential to the invention and thus should not be taken in their restrictive sense, i.e. should not be taken as to limit the scope of the present invention. It is to be understood that other suitable components and cooperation thereinbetween, as well as other suitable geometrical configurations may be used for the system and corresponding parts according to the present invention, as will be briefly explained hereinafter and as can be easily inferred herefrom, without departing from the scope of the invention.

### LIST OF NUMERICAL REFERENCES FOR SOME OF THE CORRESPONDING OPTIONAL COMPONENTS ILLUSTRATED IN THE ACCOMPANYING DRAWINGS

10. system
12. evacuation pit
14. biomass (ex. “organic matter”)
16. biogas
18. opening
18a. lower opening
18b. upper opening
20. digestion reservoir
22. inlet (of digestion reservoir)
24. outlet (of digestion reservoir)
26. hydrolysis pit
28. inflow pipe (to hydrolysis pit)
30. upright or vertical divider (or simply “divider”)
32. passageway (along digestion path)
32a. first passageway
32b. second passageway
32c. third passageway
32d. fourth passageway
40. mixing assembly (or “transferring” assembly—ex. an “agitation unit”)
42. vertical structure (or vertical “chimney”)
44. agitation unit (or simply “agitator”)
46. mixing device (ex. propeller, etc.)
48. cover
50. energy storage unit
52. hot water pit
54. burner
56. heat exchanging circuit
60. greenhouse
62. pipeline
64. feeding pipe
66. pump
68. base (of digestion reservoir)
70. temperature-regulating system
72. dome (of reservoir)

Broadly described, the present invention relates to a system 10 and a method for producing biogas 16 from decaying biomass 14, hereinafter referred to also as “organic matter” 14. The words “produce” and “producing” as used herein refer to the creation, generation, promotion, emission, etc. of biogas 16 from the decaying organic matter 14. Said differently, the decay of organic matter 14 results in the release of biogas 16, and it can therefore be said that the biogas 16 is “produced” from the decaying organic matter 14. The term “biogas” 16 can refer to any gaseous or semi-gaseous fluid produced by the breakdown of the organic matter 14, in aerobic, anaerobic, and/or any combination of these conditions. In some optional configurations, the biogas 16 is largely composed of methane. The term “decay” as used herein can refer to the rot, decomposition, break down, deterioration, dilapidation, disintegration, etc. of the organic matter 14 through natural or assisted processes. The expression “biomass” or “organic matter” 14 as used herein refers to any material that has come from a living and/or once-living organism and/or organisms, and which is capable of decay. As such, the organic matter 14 can be plant and/or animal-based biomass 14, manure, sewage, municipal waste, green waste, plant material, crops, etc. The organic matter 14 can be solid, gaseous, liquid, and/or any of these states of matter combined. In some optional configurations of the present invention, the organic matter 14 being used is agricultural animal manure, and some of these configurations may be described with reference to such manure. It is understood that such a description does not limit the use of the system or method to manure in particular.

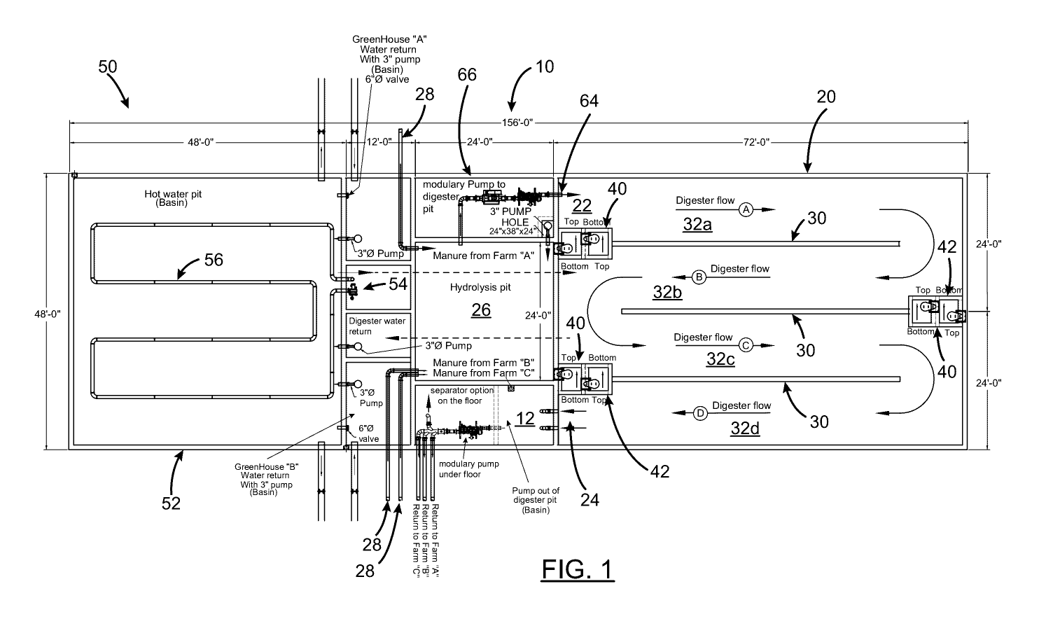

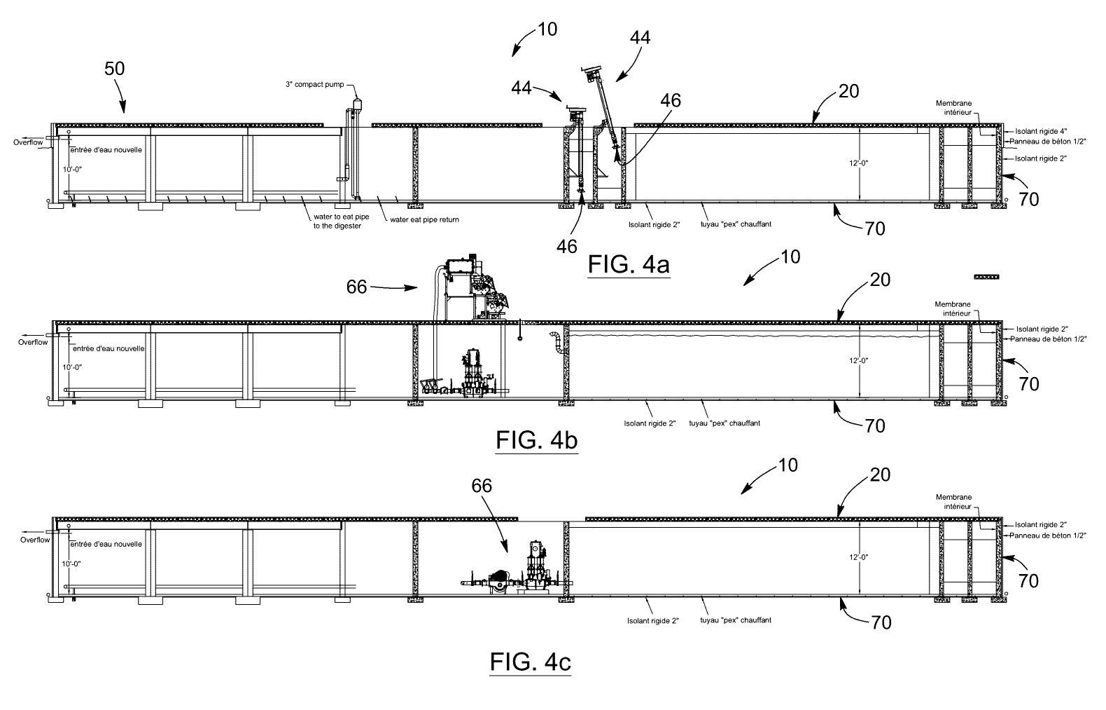

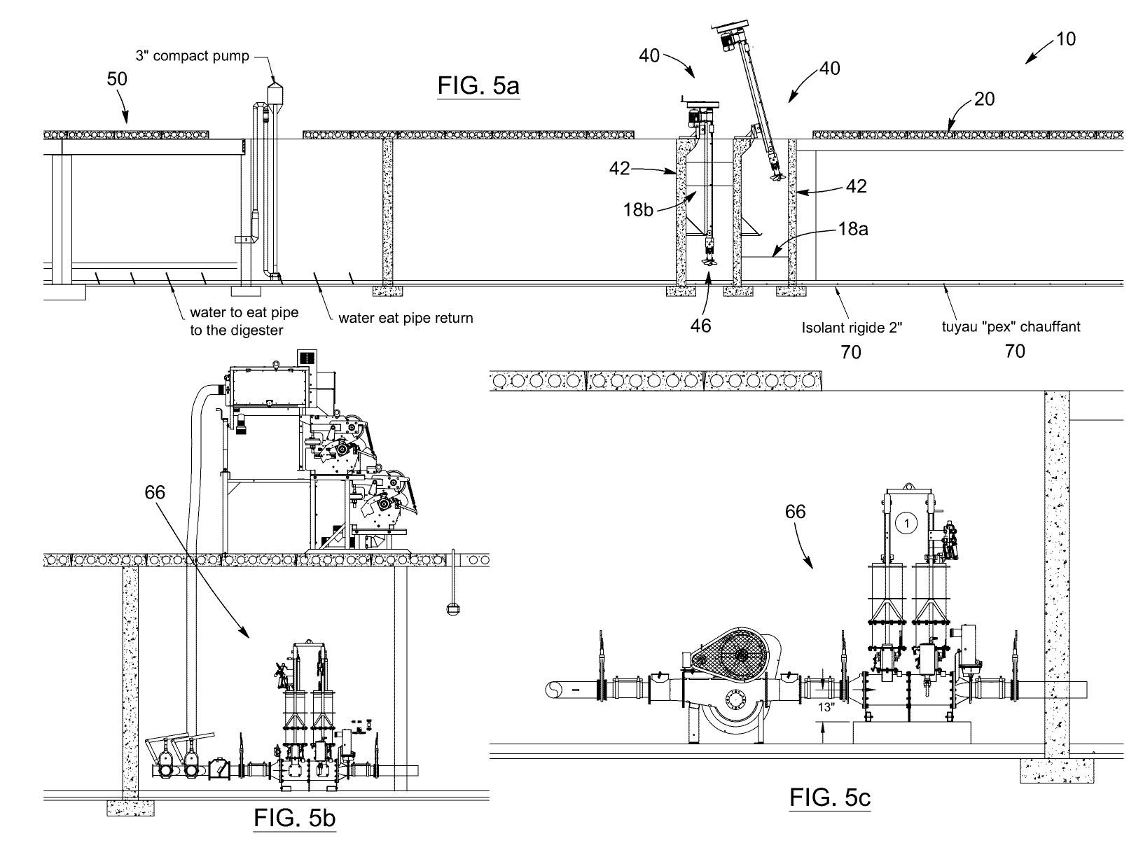

Referring to FIGS. 1, 4, and 5, the system 10 has a reservoir 20, which may be sealed. The term “sealed” as used to describe the reservoir 20 means that the reservoir 20 is substantially impermeable to fluids contained therein. Some examples of such fluids include liquid portions of the organic matter 14, such as agricultural manure in liquid form (i.e. slurry). These liquid portions are contained within the reservoir 20, and the reservoir 20 is thus impermeable because it does not allow said liquid to escape. Another example of such fluids is the biogas 16. The reservoir 20 is intended to prevent the biogas 16 from escaping, thereby allowing it to accumulate above the organic matter and be collected. Therefore, the reservoir 20 can take the form of, or be equipped with, a leak-proof dome 72 in which the biogas accumulates. The reservoir 20 can also be impermeable to solid organic matter 14. The reservoir 20 is any volume within which fluid and/or solids can collect. As such, the reservoir 20 can be a basin, container, repository, holder, pond, pool, receptacle, reserve, etc. which stores the organic matter 14 and allows it to be digested so as to produce biogas.

The reservoir 20 includes an inlet 22. The organic matter to be processed is brought into the reservoir 20 through the inlet 22. As such, the inlet 22 can be any conduit, pipe, opening, aperture, etc. The inlet 22 can also include a pump 66 and/or other pressurized unit which allows the reservoir 20 to receive the organic matter under pressure, or from a fluid circuit. Consider now the example where the organic matter is agricultural liquid manure. In such an optional configuration, one farm or many farms transfer their liquid manure to a tempering reservoir, such as a hydrolysis pit 26, for example, via a series of inflow pipes 28. There, the manure is allowed to settle, and the manure temperature can be raised to 100° F. and the acidity level (PH) can be verified, adjusted, etc. Once ready, the liquid manure is then transferred to the main reservoir 20, which is known as the digestion reservoir 20 or “digester pit”. From the hydrolysis pit 26, the manure enters the digestion reservoir 20 via the inlet 22.

The reservoir 20 also includes an outlet 24. The outlet 24 conveys the decayed organic matter from the reservoir 20. As with the inlet 22 described above, the outlet 24 can include a pump and/or pressurized unit to convey the decayed organic matter from the reservoir 20. As explained below, the passage of the decaying organic matter through the reservoir 20 results in the production of biogas. “New” organic matter (i.e. organic matter just introduced through the inlet 22 and/or organic matter that has not significantly decayed) thus moves through the reservoir 20. As the new organic matter decays, it becomes “older” (i.e. is not likely to decay further or as much), and is thus less likely to produce biogas. Advantageously, the outlet 24 of the reservoir 20 allows for this older organic matter to be evacuated, expelled, removed, etc. from the reservoir 20, thereby creating space within the reservoir 20 for more productive new organic matter to be introduced into the system 10.

The organic matter is in motion within the reservoir 20, such that it is conveyed from the inlet 22 to the outlet 24. Such motion and/or conveyance can be achieved through many different techniques. Indeed, one example of such a technique involves having a digestion reservoir 20 with a downwardly-sloping base 68, that is, angling a base 68 of the reservoir 20 towards the outlet 24 such that the organic matter flows under gravity from the inlet 22 to the outlet 24, which advantageously reduces the complexity of the system 10. Alternatively, if a faster motion and/or conveyance is desired, a mechanical conveyance, such a pump, pressurized unit, a conveyor, etc. can be used. In yet another possible alternative, the mere addition of organic matter at the inlet 22 can propel organic matter already in the reservoir 20 toward the outlet 24. Of course, many other such techniques can be used to convey the organic matter, and these techniques are within the scope of the present invention. In some optional configurations, a hot-water circulation circuit and/or other temperature-regulating system 70 (i.e. a temperature-“maintaining” system 70 or temperature-“adjusting” system 70) is provided around and/or under the hydrolysis pit 26 and/or digester reservoir 20, such as in the wall(s) and/or floor(s) of these structures. Temperature-regulating can be done “actively” by means of heating/cooling systems, or can be simply done “passively” by means of insulating panels, for example. This advantageously allows for the maintenance of the temperature of the digesting organic matter at an optimal level of around 100° F., for example. The energy required for this temperature maintenance can advantageously be provided by burning the produced biogas, for example.

The system 10 also includes at least one vertical divider 30, which can be affixed to the base of the reservoir 20. The divider 30 can be any wall, partition, barrier, impediment, etc. which extends in a substantially upright manner (such as vertically from the base, for example), and which divides the reservoir 20 into passageways 32. Each passageway 32 can be any aisle, alley, couloir, path, etc. along which the organic matter can be conveyed, and which alone or in conjunction with other such passageways 32, takes the organic matter from the inlet 22 to the outlet 24. As such, the passageways can be of any dimension, shape, and/or configuration which allow such functionality.

One optional configuration of the pathways 32 is shown in FIG. 1. The passageways 32 being exemplified are “rectangular” (although various other possible geometrical configurations and/or dispositions are within the scope of the present invention) and extend alongside each other such that the end of one passageway 32 exits into the entrance of the subsequent, adjacent passageway 32. It is apparent that the passageways 32 are not limited to such a configuration, and can take any other configuration, shape, form, etc. depending on the following non-restrictive list of factors: available area and/volume of reservoir 20, the expected processing rate of organic matter, the nature of the organic matter being processed, etc. Indeed, another possible configuration of the passageways 32 can be a series of concentric circles, where new organic matter enters the inlet 22 of an outermost circular passageway 32 and is conveyed while it decays through the series of inner concentric circular passageways 32 before exiting through the outlet 24 near the shared center point of the concentric circles. Thus, it is understood that the expression “extends alongside” as used to describe the orientation of the passageways 32 refers to any configuration of the passageways 32 that allows them to remain, at least in part, adjacent and/or in proximity to one another.

In the optional configuration shown in FIG. 1, each divider 30 after the first divider 30 adds another rectangular passageway 32. Therefore, the three dividers 30 shown in FIG. 1 provide four passageways 32 within the enclosed reservoir 20. Furthermore, each divider 30 can divide the reservoir 20 into first and second passageways 32. It is understood that the expressions “first” and “second” as used herein do not limit the number of passageways 32 to only two, nor do they require that each divider 30 create two passageways 32.

The dividers 30 and/or the system 10 may include “mixing” or “transferring” assemblies 40, herein referred to also as agitation units 40. The agitation units 40 can be used to maintain mixing of the organic matter, thereby preventing a hardening and/or drying out of its upper surface which may prevent the emission of biogas. The agitation units 40 can also be used to add older organic matter to the new organic matter, thereby advantageously transferring the beneficial bacteria from the older organic matter to the new and thus accelerating activation of the new organic matter. As exemplified in FIG. 1, each agitation unit 40 can be installed at an end of each divider 30 such that it performs the above-mentioned functions on the organic matter before it enters a different passageway 32.

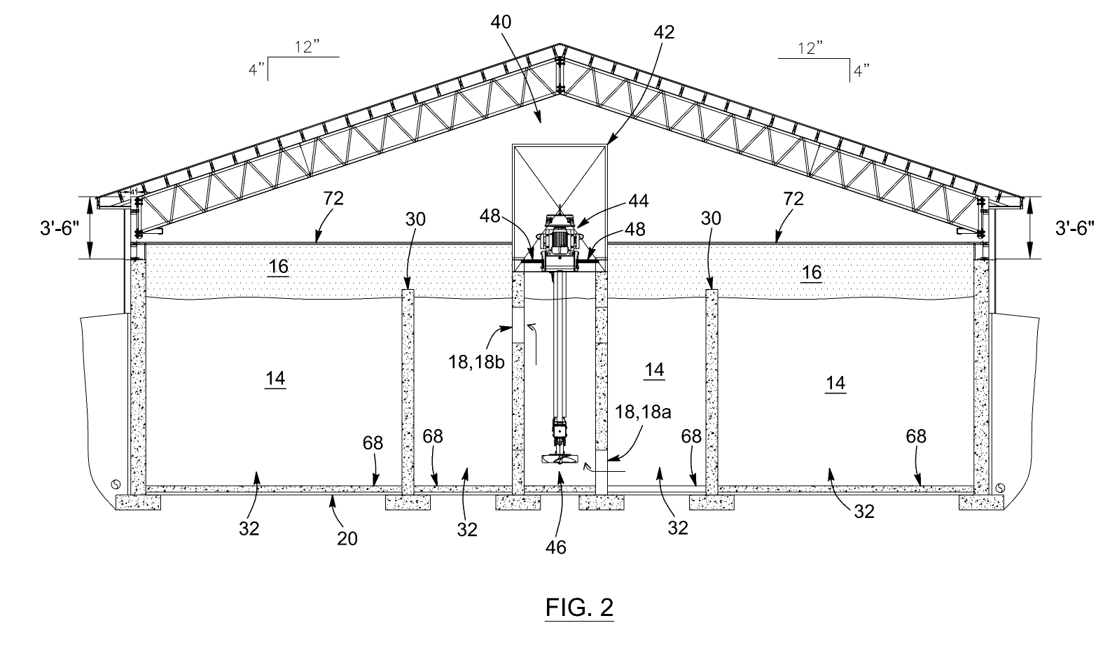

In one optional configuration, and as exemplified in FIG. 2, each agitation unit 40 may include a vertical chimney 42 installed at strategic locations in the reservoir 20, such as at the ends of the dividers 30. Each vertical chimney 42 can be equipped with an agitator 44 and/or any other similar agitating device that also acts as a transferring/mixing device. The agitator 44 can include a mixing device 46, such as a rotating propeller, which agitates the organic matter 14 so as to prevent it from solidifying and thus forming a barrier to the release of the biogas 16. The mixing device 46 can also create a force, pressure, impetus, etc. that is intended to transfer the organic matter 14 from a lower portion of one passageway 32 to the upper portion of an adjacent passageway 32, as exemplified by the arrows in FIG. 2 showing the direction of movement of the organic matter 14. This transfer can occur through openings 18 in the dividers 30. In one example of such a transfer, the organic matter 14 is brought, pulled, forced, etc. through a lower opening 18a by the mixing device 46, up through the vertical chimney 42, and then into an upper portion of the organic matter 14 through an upper opening 18b. As exemplified in the accompanying drawing, the system may comprise pair of vertical chimneys 42 at different locations along the path of the digestion reservoir.

Each vertical chimney 42 can consist of two parts. A first part transfers older/digested organic matter 14 from the base of the reservoir 20 in one passageway 32 on one side of its divider 30 to the top of the new organic matter 14 in the preceding adjacent passageway 32 on the other side of its divider 30, as previously explained. Of course, the operation of the vertical chimney 42 is not limited to a bottom-to-top transfer. Indeed, a second part of the vertical chimney 42 can transfer not-yet-fully digested organic matter 14 from the top of the reservoir 20 in one passageway 32 on one side of its divider 30 to the bottom of the new organic matter 14 in a preceding adjacent passageway 32 on the other side of its divider 30. The advantage of having passageways 32 that extend alongside one another can thus be clearly understood. This agitation and transferring of organic matter 14 advantageously allows for a mixture of beneficial bacteria, thus allowing the new organic matter 14 to acquire the beneficial bacteria sooner in the process when compared to conventional systems, and thus further assisting production of the biogas. Of course, the configuration and operation of the vertical chimneys 42 is not limited to the description given herein, and the vertical chimneys 42 can operate differently than as described provided that they can agitate the organic matter 14 and effect a transfer of beneficial bacteria from one passageway 32 to another passageway 32 (whether an adjacent passageway 32, or a more remote passageway 32). Indeed, mixing assemblies 40 according to the present system are not necessarily limited to extending between immediately adjacent passageways 32, in that, for example, the system 10 contemplates the idea of transferring organic matter from one of the more “downstream” passageways 32, where “older” biomass rich in digestion bacteria can be found, to one of the more “upstream” passageways 32 where new biomass is found and which could benefit from such advantageous digestion bacteria so as to increase overall production of biogas for a same given time period or cycle (ex. 30 days), and for a given volume of biomass 14 inside the digestion reservoir 20, something that is not possible with conventional systems.

In some optional configurations of the present system 10, the vertical chimney 42 can have a cover 48 that can be opened on top of each individual part of each vertical chimney 42, thereby giving direct access to the interior of the vertical chimney 42. The cover 48 can be of any suitable size and configuration, and can limit the size of the opening in the vertical chimney 42 to only a small area such that the biogas 16 is prevented from escaping to atmosphere because the level of the organic matter 14 is higher than the level of the upper opening 18a, as exemplified in FIG. 2, as but one possible configuration of the vertical chimney 42. The cover 48 also facilitates access so as to inspect, remove, maintain and/or reinstall the agitation unit 40, and/or other aspects of the system 10, without interrupting or impacting the digestion process. Advantageously, such access can increase the total production of biogas 16, and thus increase profitability and avoid significant downtime periods in energy production.

The technique for agitating/transferring the digesting organic matter 14 is not limited to the technique described above. Similarly, the agitation unit 40 is not limited to the vertical chimney 42 described, and can be any other mechanical device providing the same and/or similar functionality. Indeed, the techniques described above for opening the vertical chimneys 42 and/or for transferring digesting organic matter 14 from one passageway 32 to another is not limited to what is described above, and vertical chimneys 42 with intake and outlet opening(s) 18 that are on same side so as to only agitate in the same passageway 32 is also part of the present system 10.

Indeed, in most conventional digesters, such as “plug flow” digesters for example, sediments tend to accumulate and solidify at the bottom of the reservoir over time, and these ever-increasing deposited layers tend to reduce the effective flow rate of biomass along the reservoir, in some cases, causing the digester to become very inefficient or even inoperable. The provision of a mixing assembly 40 according to the present system 10, at one or several discrete locations along the path of the digestion reservoir 20, with corresponding optional components (agitation units 44, openings 18, etc.) enables to prevent or at the very least minimize the occurrence of sediments depositing at the bottom of the reservoir 20 along the path, via localized agitations of and/or mixing between passageways 32, thereby enabling an effective flow rate of biomass 14 along the entire digestion reservoir 20 and corresponding system 10.

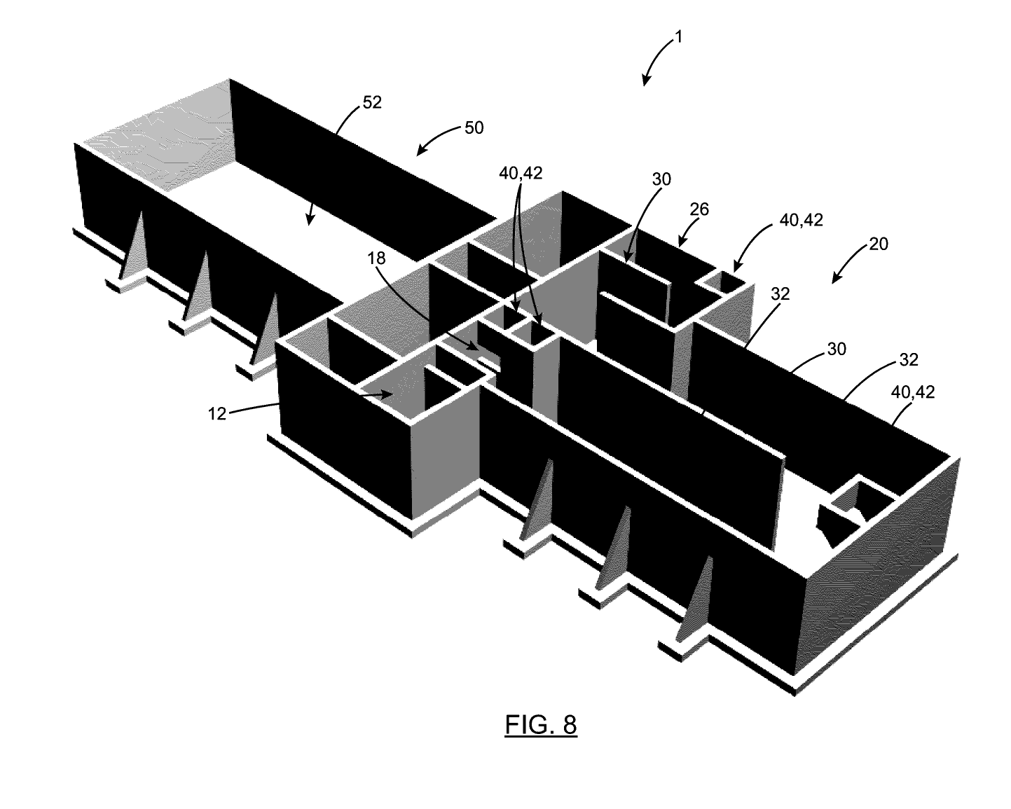

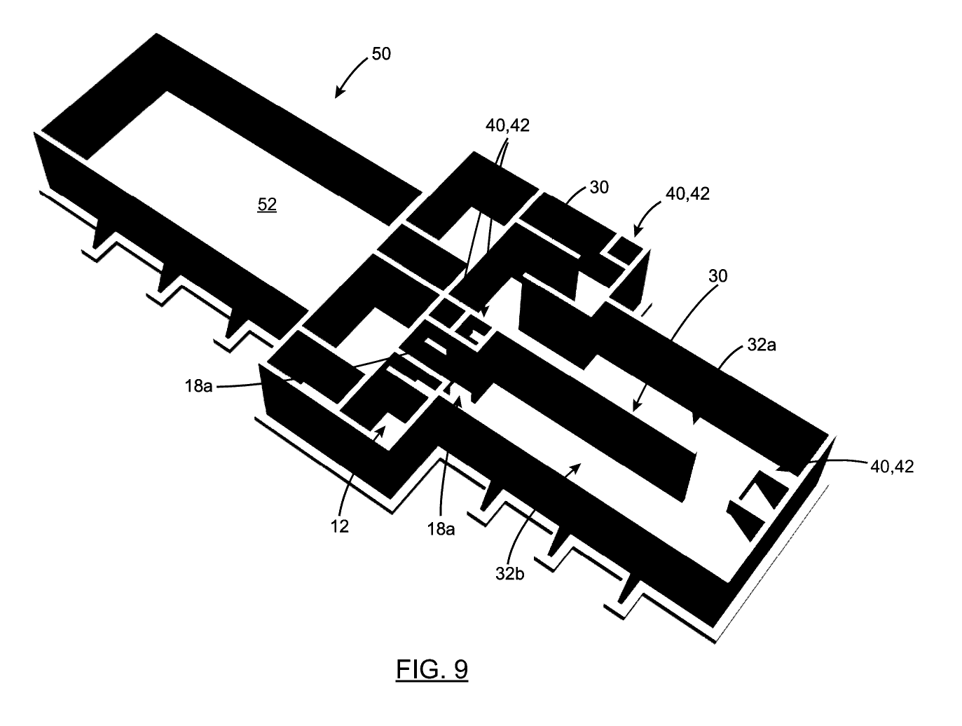

It is of course understood that the size, configuration, and/or capacity of the system 10 and/or its features and components can be adapted to specific on-site requirements and is not limited to a particular size, configuration, and/or capacity. Furthermore, the system 10 and/or its features and components can be applied separately to upgrade an existing system for generating biogas 16 or for burning combustibles. Also, the use of dividers 30 and/or of mixing assemblies 40 according to the present system is not limited to the digestion reservoir 20, and may be employed in other parts of the system 10 (ex. hydrolysis pit 26, etc.), as exemplified in FIGS. 8 and 9.

Having described some of components of the system 10, the production of biogas by conveyance through said system 10 will now be described.

Returning to FIG. 1, in one optional configuration, the reservoir 20 is configured to digest the organic matter in about 30 days. The series of passageways 32 force the digesting organic matter forward when new organic matter is added to the reservoir 20 at the inlet 22. Older digested organic matter is thus expelled from the system 10 at the outlet 24, and can be returned by gravity or under pressure to an evacuation pit 12 for its transfer to to another application. It is thus apparent how digestion time is controlled in the reservoir 20 by the addition of new organic matter and the expulsion of older organic matter, which has been allowed to remain in the reservoir 20 (and thus produce biogas) for a relatively long period. The digestion time can be varied as required by simply varying the rate of addition of new organic matter, for example.

Biogas is generated as the organic matter decays while being conveyed through the passageways 32. To “kick-start” digestion, bacteria can be added to the organic matter. The bacteria can be any microorganism which releases biogas as it consumes the organic matter. The addition of bacteria can be performed manually, at the inlet 22, for example. Alternatively, and advantageously, the digestion reaction of the organic matter can be started by adding the bacteria which is already found in relatively large numbers in the organic matter in adjacent and/or passageways 32 in front (i.e. “downstream”) of the organic matter. Thus, a part of older digesting organic matter already containing bacteria can be conveniently and advantageously transferred from the end of one passageway 32 to the beginning of another passageway 32. In the optional configuration shown in FIG. 1, the transfer of older organic matter can occur from passageway 32 “B” to the beginning of passageway 32 “A”, thereby activating digestion of the new organic matter. This transfer can be affected with the agitation units 40 described above. At the same time, the local movement of the agitation units 40 can provide a mixing effect to maintain a smoothly-moving organic matter, thereby stimulating biogas production and avoiding creation of a solid dry layer on top of the organic matter that would prevent biogas from escaping the digesting organic matter.

It can thus be appreciated that the system 10 can allow for a controlled mixing and digesting of older and new organic matter. At the end of passageway 32 “B”, the digesting organic matter can be about 15 days old, for example. Thus, the transfer of organic matter between passageways 32 can be done from one passageway 32 to its immediately preceding and/or adjacent passageway 32, thereby avoiding transferring organic matter from the outlet 24 to the inlet 22 which may be too decayed to be kept in the reservoir 20. In some optional configurations, the transferring/mixing/agitation action can be controlled in frequency and time to facilitate mixing and agitation.

In some optional configurations, the system 10 can have an energy storage unit 50. The biogas produced in the reservoir 20 has relatively high energy potential due to its chemical energy. In order to take advantage of such energy, it is often desirable to store it such that it can be used at a later time. This can be achieved in many ways. For example, the energy of the biogas can be converted to electrical energy, such as by combusting the biogas in an electrical turbine. Alternatively, the energy of the biogas can be stored as thermal energy. This accumulated thermal energy can then later be used for any application needing a reliable supply of heat energy.

The energy storage unit 50 can include a hot water pit 52, which can contain a large volume of a liquid for storing the heat energy, such as water. The energy storage unit 50 can also include a burner 54, which burns the accumulated biogas and transfers the heat generated thereby to the water of the hot water pit 52. This transfer of heat from the burner 54 to the hot water pit 52 can be performed by using a heat exchanging circuit 56, which can be a series of pipes forming a hot gas circuit originating at the burner 54 and ending at an outside evacuation. These pipes can transfer the hot gas from the exhaust of the burner 54 through the water of the hot water pit 52, thereby heating the water while the pipes are simultaneously cooled by the water. Thus, calorific energy is accumulated in the water as water temperature rises. In order to advantageously preserve as much calorific energy as feasible, the walls of the hot water pit 52 can be insulated (with insulating panels, for example), thereby helping to maintain a high efficiency level. Consider the example of a hot water pit 52 which has the following dimensions 12 ft.×24 ft.×24 ft., which provides a volume of 6,912 cubic ft. In such a hot water pit 52, the accumulated thermal energy can be about 1,400,000 BTU/° F.

In yet another example, consider the biogas generated from a system 10 calibrated for a farm having about 400 cows. Such a system 10 may produce enough heat energy to maintain a hot water pit 52 of 7,000 cubic ft. at a temperature of 130° F. for a significant period of time. This hot water may maintain a difference of about 60° F. (for example) over a reference such as 70° F., for ex ample. The accumulated available calorific energy accumulated in this hot water can thus be a minimum of about 84,000,000 BTU.

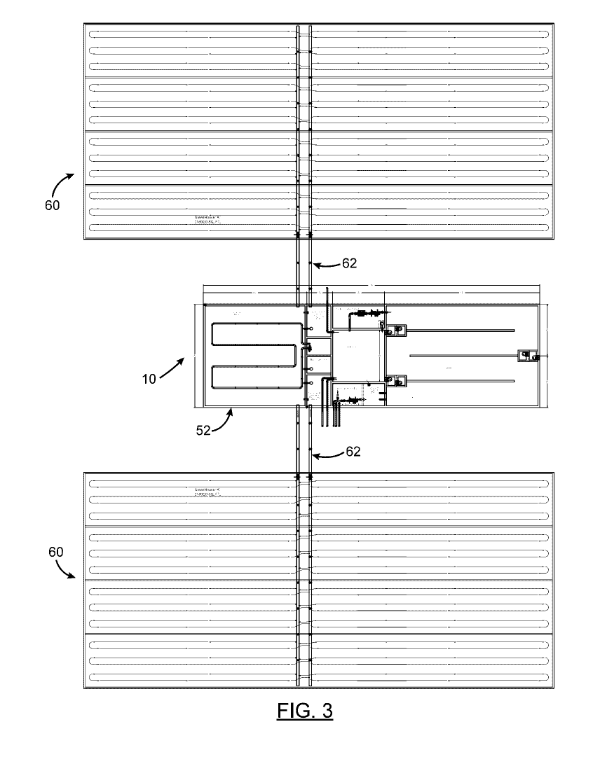

Once the system 10 has generated a reliable and usable energy source (i.e. thermal energy), such energy can be used as desired. Such heat can be used to supply an application that requires heat input, and which could thus avoid energy losses associated with the transformation of energy. Referring to FIG. 3, one such application could be a greenhouse 60, for example.

At least one greenhouse 60 can be built near the system 10 and/or near the hot water pit 52. The greenhouse 60 could be floor-heated by using the thermic energy accumulated in the water of the hot water pit 52. Thus, the use of this thermic energy without transforming it into another form of energy can increase considerably the efficiency and the total available energy. In one example of how such a greenhouse 60 could be installed, a hot water pipeline 62 can be installed in the floor of the greenhouse 60. The pipeline 62 can be connected to the hot water pit 52 so as to transfer the heat of the hot water to the floor of the greenhouse 60. Thus, and as mentioned, relatively little energy is lost because no transformation of energy occurs. Thermal energy that was in the water is now in the greenhouse 60. It is understood that the transfer of thermal energy from the hot water pit 52 is not limited to being performed by pipes or the pipeline 62, and that any other suitable technique to transfer the heat energy, with or without transforming it, is within the scope of the invention.

Consider the example of a greenhouse 60 installed in a northern climate such as that found in the province of Québec, Canada, for example. The daily heat input required for a 1-acre greenhouse 60 during the coldest period of the year (January) is about 23,000,000 BTU. The system 10 calibrated for the manure of about 400 cows can maintain a hot water pit of about 7,000 cubic ft. at about 130° F. and thus can have enough accumulated energy to maintain the required temperature of a 1-acre greenhouse 60 for 2 days of this coldest period. Since the system 10 can allow maintenance without interruption, operators of the greenhouse 60 thus have a reliable source of heat.

It is of course understood that the thermal energy stored by the energy storage unit 50 is not limited to being used in a greenhouse 60, and that the thermal energy can be used for any application where a reliable source of heat is required. Also, one example of such an application relates to food production and food preservation before delivery. The use of thermal energy may not be necessary in warmer months of the year, or may be limited to simply prevent food from freezing. In summer months, the biogas can be used to create a cold environment using technology such as a propane refrigerator adapted to biogas. During transition periods (i.e. spring and fall), the use of the biogas can be split between these two applications. The versatility of the produced biogas thus advantageously allows for efficient use of the biogas yearlong, which can increase profitability of the system 10, and that of farm(s) or other facilities associated to the system 10.

Returning to FIG. 1, and according to one aspect of the present invention, there is provided a method for producing biogas from decaying organic matter. The method includes the steps of introducing the organic matter to the reservoir 20. The method also includes the step of displacing the decaying organic matter within the reservoir 20 in adjacent passageways 32, and the decaying organic matter produces biogas while being displaced. In some optional configurations, the method also includes the steps of introducing beneficial bacteria into non-decayed organic matter so as to activate the reaction of the organic matter. Such an introduction of bacteria can be accomplished by agitating and/or transferring the organic matter, as described above.

The system 10 and corresponding parts may be made of substantially rigid materials, such as metallic materials, hardened polymers, composite materials, cementitious mixture, and/or the like, as well as possible combinations thereof, depending on the particular applications for which the system 10 is intended for, and the desired end results.

As can be easily understood when referring to the accompanying drawings, the following optional components and features of the system 10 and method offer several advantages with respect to the prior art, as will be explained in greater detail hereinbelow.

Indeed, the system 10 and method described herein is an improvement over the prior art in that, by virtue of its design and components, they provide a profitable biogas system 10 that can reduce the environmental foot print of known systems, and which uses technology within the normal field of competence of a lay person, such as a farmer. It can now be understood that the system 10 is designed in a way to obtain the advantages of the prior art “infinitely mixed” and “plugged flow” systems, while eliminating or improving disadvantages of these concepts discussed above.

Furthermore, the system 10 and method allow for the production of a significant level of usable energy by eliminating many steps of energy transformation and the attendant energy losses associated therewith. Indeed, the system 10 and method allow for the direct transfer of calorific energy generated by biogas to a storage medium, such as water, for example. In can thus be appreciated that the accumulation of calorific energy allows for the storage of excess energy, which can later be made available during a period of high need. Therefore, the final total usable energy is increased significantly.

One optional aspect of the system 10, such as the vertical chimneys 42 for example, advantageously allow maintenance of the agitation units 40 without stopping biogas production, and without emitting biogas into atmosphere.

Further advantageously, in northern areas, during a significant duration of the year, fruits and vegetables come from southern areas. This equates to dependence on long transportation routes that generate pollution and increase the environmental foot print of every citizen. The use of greenhouses 60 with the present system 10 allows for the production of food locally, which procures significant environmental advantages and provides a diversified source of income for a dairy farmer, as but one example.

Although preferred embodiments of the present invention have been briefly described herein and illustrated in the accompanying drawings, it is to be understood that the invention is not limited to these embodiments and that various changes and modifications could be made without departing form the scope and spirit of the present invention, as defined in the appended claims.

1-53. (canceled)

54. A system for producing biogas from biomass the system comprising:
a digestion reservoir having a path defined by passageways along which biomass is conveyed and digested, the digestion reservoir having an inlet fluidly connected to the path for receiving biomass to be digested, and an outlet fluidly connected to the path for releasing digested biomass, the digestion reservoir being further configured for containing biogas generated from a digestion of biomass along the path; and
at least one mixing assembly located along the path, each mixing assembly being operatively connected between different passageway segments of the path so as to be able to selectively mix given biomass from one segment to another, in order to increase overall production of biogas along the path.

55. The system of claim 54, wherein the path comprises a plurality of passageways.

56. The system of claim 54, wherein the passageways are adjacent to one another.

57. The system of claim 54, wherein the passageways are rectangular.

58. The system of claim 54, wherein the passageways are defined by least one upright divider.

59. The system of claim 54, wherein each divider projects upwardly from a base of the digestion reservoir.

60. The system of claim 54, wherein the passageways are cylindrical.

61. The system of claim 60, wherein the passageways are concentric to one another.

62. The system of claim 54, wherein the digestion reservoir is substantially impermeable to fluids.

63. The system of claim 54, wherein the digestion reservoir comprises a dome for retraining biogas produced from the system.

64. The system of claim 54, wherein each mixing assembly comprises a vertical structure, the vertical structure having a first opening fluidly connected to a first given passageway of the path of the digestion reservoir, and the vertical structure having a second opening fluidly connected to a second given passageway of the path of the digestion reservoir.

65. The system of claim 64, wherein the first opening is a bottom opening and wherein the second opening is a top opening.

66. The system of claim 64, wherein the first opening is a top opening and wherein the second opening is a bottom opening.

67. The system of claim 54, wherein each mixing assembly comprises a pair of first and second vertical structures, the first and second openings of the first vertical structure being respectively bottom and top openings, and the first and second openings of the second vertical structure being respectively top and bottom openings.

68. The system of claim 67, wherein the first and second vertical structures are adjacent to one another.

69. The system of claim 64, wherein each given vertical structure comprises a cover for selectively allowing access to an inside portion of said given vertical structure.

70. The system of claim 54, wherein each mixing assembly is located at a distal end of a given divider, and wherein openings of the mixing assembly are fluidly connectable to adjacent passageways through said given divider.

71. The system of claim 54, wherein each mixing assembly comprises at least one agitation unit for locally mixing biomass being conveyed along the path and travelling about said at least one agitation unit.

72. The system of claim 71, wherein each agitation unit is provided with a mixing device.

73. The system of claim 72, wherein the mixing device is positioned adjacent to a given opening of a given mixing assembly.

74. The system of claim 54, wherein a level of biomass contained in a given passageway of the digestion reservoir is maintained above a corresponding opening of the least one mixing assembly associated to said given passageway so as to prevent biogas from escaping the system via said at least one mixing assembly.

75. The system of claim 54, wherein a level of biomass contained in the digestion reservoir is maintained above an uppermost opening of the least one mixing assembly of the system so as to prevent biogas from escaping the system via said at least one mixing assembly.

76. The system of claim 54, wherein the system comprises a hydrolysis pit for providing the digestion reservoir with biomass.

77. The system of claim 54, wherein the system comprises at least one pump for pumping biomass from the hydrolysis pit to the digestion reservoir.

78. The system of claim 54, wherein the biomass is agricultural liquid manure provided by at least one neighboring farm operatively connected to the hydrolysis pit of the system via at least on inflow pipe.

79. The system of claim 54, wherein the system comprises an evacuation pit for receiving digested biomass from the outlet of the digestion reservoir.

80. The system of claim 54, wherein the system comprises at least one pump for pumping digested biomass out from the system.

81. The system of claim 54, wherein the digestion reservoir comprises a downwardly-sloping base, and wherein biomass is conveyed along the path of the digestion reservoir by gravity via said downwardly-sloping base.

82. The system of claim 54, wherein biomass is conveyed along the path of the digestion reservoir via an inflow of new biomass at the inlet of the digestion reservoir and via an outflow of old biomass at the outlet of the digestion reservoir.

83. The system of claim 54, wherein biomass is conveyed along the path of the digestion reservoir via a mechanical conveyance.

84. The system of claim 54, wherein the system comprises a temperature-regulating system provided about the digestion reservoir.

85. The system of claim 76, wherein the system comprises a temperature-regulating system provided about the hydrolysis pit.

86. The system of claim 54, wherein biomass is digested along the path of the digestion reservoir by a natural process.

87. The system of claim 54, wherein biomass is digested along the path of the digestion reservoir by an assisted process.

88. The system of claim 54, wherein digestion bacteria are introduced into the digestion reservoir.

89. The system of claim 54, wherein biomass is digested along the path of the digestion reservoir during a period of about 30 days.

90. The system of claim 54, wherein the system is used to produce methane.

91. The system of claim 54, wherein biogas produced from the system is recuperated and converted into electrical energy.

92. The system of claim 54, wherein biogas produced from the system is recuperated and stored into thermal energy.

93. The system of claim 54, wherein the system comprises an energy storage unit for storing thermal energy.

94. The system of claim 93, wherein the energy storage unit comprises a burner for burning biogas produced by the system.

95. The system of claim 54, wherein the system comprises a hot water pit for receiving heat from burnt biogas via a heat exchanging unit conducting said burnt biogas.

96. The system of claim 95, wherein the hot water pit is operatively connected to at least one neighboring greenhouse.

97. The system of claim 96, wherein the least one neighboring greenhouse has a floor circuit heated by means of hot water provided by the hot water pit.

98. The system of claim 95, wherein the system comprises a temperature-regulating system provided about the hot water pit.

99. A kit with components for assembling a system of claim 54.

100. A method for producing biogas from biomass, the method comprising the steps of:
a) providing a system of claim 54;
b) conveying and digesting biomass along the path of the digestion reservoir; and
c) transferring given biomass from one passageway to another in order to increase overall production of biogas along the path.

101. The method of claim 100, wherein the method comprises the step of agitating biomass at discrete locations along the path.

102. The method of claim 100, wherein the method comprises the step of introducing beneficial bacteria into the digestion reservoir.

103. The method of claim 100, wherein the method comprises the step of regulating the temperature of the digestion reservoir.

104. The method of claim 100, wherein the method comprises the step of maintaining the biomass at a temperature of about 100° F.

105. The method of claim 100, wherein step b) comprises the step of digesting biomass during a period of about 30 days.

106. The method of claim 100, wherein the method comprises the step of recuperating biogas produced by the system and converting it into usable energy.
